(19) United States
(12) Patent Application Publication
(10) Pub. No.: US 2023/0277790 A1
(43) Pub. Date: Sep. 7, 2023

(54) ENDOTRACHEAL INTUBATION FOR PREVENTING INJURY TO THROAT, VOCAL CORDS, AND TRACHEA

(71) Applicant: National Taichung University of Science and Technology, Taichung (TW)

(72) Inventors: Bor-Tsang WU, Taichung (TW); Tien-Chi HUANG, Taichung (TW); Shin-Da LEE, Taichung (TW); Su-Chi LIN, Taichung (TW); Po-Chi CHAN, Taichung (TW); Yu-Lin CHAN, Taichung (TW)

(21) Appl. No.: 18/178,277

(22) Filed: Mar. 3, 2023

(30) Foreign Application Priority Data
Mar. 4, 2022 (TW) 111108014

(51) Int. Cl. A61M 16/04

(52) U.S. Cl. CPC A61M 16/0488; A61M 16/0402

(57) ABSTRACT

The invention is an endotracheal intubation for preventing injury to throat, vocal cords and trachea. The endotracheal intubation comprises a hollow intubation body with a variable outer diameter. The hollow intubation body comprises a front end and a tail end, an inner intubation wall and an outer intubation wall, and having a retracted state with a smaller outer diameter and an expanded state with a larger outer diameter. When the endotracheal intubation inserted into a patient’s trachea needs to be replaced, as long as the hollow intubation body of the new endotracheal intubation is in the retracted state, it can be inserted into the hollow intubation body to be replaced, and when reaching the position, the hollow intubation body to be replaced is withdrawn, and finally the new hollow intubation body is changed into the expanded state, i.e. the replacement operation of the endotracheal intubation is completed.

### FIELD OF THE INVENTION

The present invention relates to an endotracheal intubation, and more particularly to an endotracheal intubation for preventing injury to throat, vocal cords, and trachea.

### BACKGROUND OF THE INVENTION

Endotracheal intubation is a device to assist oxygen breathing. For insertion into a patient’s trachea, the main purpose is to expand the trachea to ensure smooth breathing, and can be used for sputum extraction, drug administration and other treatment methods. For patients with severe pneumonia, it is a life-saving means to assist oxygen breathing. Conventional endotracheal intubation constructions are shown in U.S. Publication No. US20100258134 A1 entitled “Endotracheal intubation having improved suction lumen”. The primary construction comprises an intubation body and a joint end and a front end with a chamfered end face which are located on both ends of the intubation body. The front end is inserted into the patient’s trachea and the joint is for connection to an oxygen supply device.

In order to avoid the occurrence of infection, the patients using endotracheal intubation must replace it after a certain period of use. However, the patients who will use endotracheal intubation usually cannot be short of the oxygen supply device for a long time. Therefore, the speed of replacing endotracheal intubation shall be fast, which results in that the throat, vocal cords and trachea are easily injured due to friction when replacing endotracheal intubation. Once the throat, vocal cords and trachea are injured, it not only causes discomfort to the patient, but also increases the chance of infection.

### SUMMARY OF THE INVENTION

It is a primary object of the present invention to disclose an endotracheal intubation that can be replaced without causing injury to the throat, vocal cords and trachea.

To achieve the above-mentioned object, the present invention is an endotracheal intubation for preventing injury to the throat, vocal cords and trachea, comprising: a hollow intubation body of variable outer diameter dimension, the hollow intubation body comprising a front end, a tail end, an inner wall and an outer wall, wherein the front end and the tail end are located at opposite ends of the hollow intubation body, the inner wall and the outer wall are located at opposite inner and outer sides of the hollow intubation body, and the hollow intubation body has a retracted state with a smaller outer diameter and an expanded state with a larger outer diameter.

Accordingly, the endotracheal intubation of the present invention can be mounted in a trachea of a patient, and when the endotracheal intubation needs to be replaced by a new endotracheal intubation, the hollow intubation body of the old endotracheal intubation is not removed, but the hollow intubation body of the new endotracheal intubation is in the retracted state and directly penetrates into the old hollow intubation body. By using the old hollow intubation body as a protective layer to avoid injury to the trachea. When the new hollow intubation body of the endotracheal intubation reaches the position, the old hollow intubation body is withdrawn, and the new hollow intubation body is changed to the expanded state so as to complete the replacement operation of the endotracheal intubation.

### BRIEF DESCRIPTION OF THE DRAWINGS

FIG. 1 is a schematic diagram showing the overall appearance and structure of an endotracheal intubation of the present invention.

FIG. 2 is a schematic cross-sectional diagram of a hollow intubation in a retracted state according to the present invention.

FIG. 3 is a schematic cross-sectional diagram of a hollow intubation in an expanded state according to the present invention.

FIG. 4 is a schematic cross-sectional diagram of a hollow intubation in a retracted state according to another embodiment of the present invention.

FIG. 5 is a schematic cross-sectional diagram of a hollow intubation in an expanded state according to another embodiment of the present invention.

FIG. 6 is a schematic diagram of the use and replacement of an endotracheal intubation according to another embodiment of the present invention.

### DETAILED DESCRIPTION OF THE PREFERRED EMBODIMENTS

With regard to the detailed technical contents of the present invention, reference is now made to the following description taken in conjunction with the accompanying drawings:

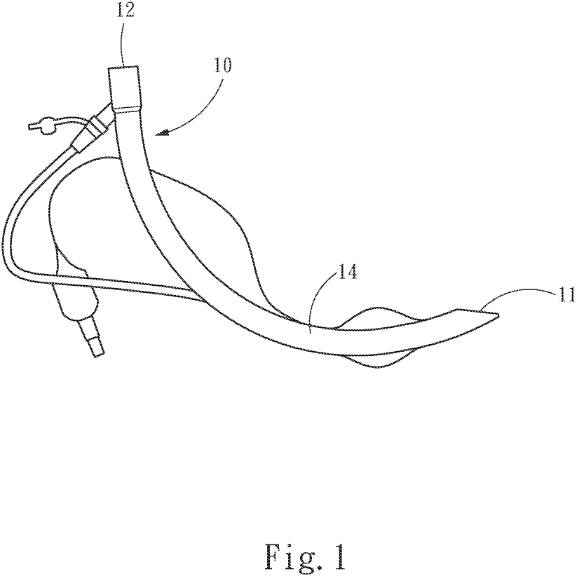

With reference to FIGS. 1, 2 and 3, the present invention is an endotracheal intubation for preventing injury to the throat, vocal cords and trachea comprising a hollow intubation body 10 of variable outer diameter dimension. The hollow intubation body 10 includes a front end 11, a tail end 12, an inner wall 13 and an outer wall 14, wherein the front end 11 and the tail end 12 are located at two opposite ends of the hollow intubation body 10, the inner wall 13 and the outer wall 14 are located at two opposite inner and outer sides of the hollow intubation body 10, and the hollow intubation body 10 includes a retracted state with a smaller outer diameter and an expanded state with a larger outer diameter.

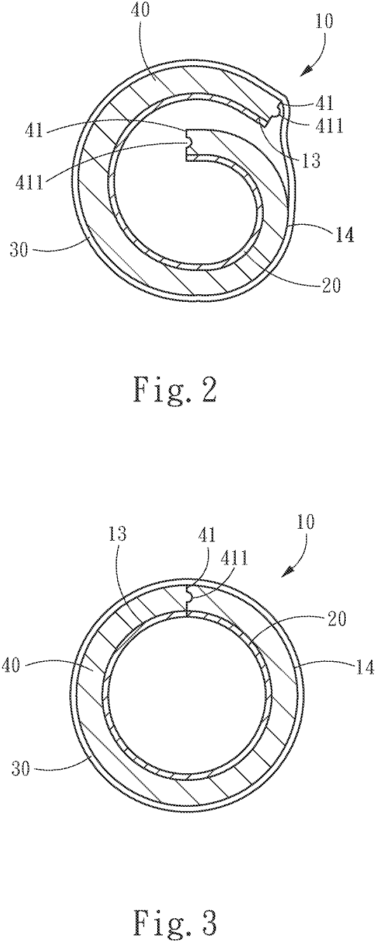

As shown in FIGS. 2 and 3, in one embodiment, the hollow intubation body 10 is a longitudinally sectioned intubation piece 40 in a curve shape, and the longitudinally sectioned intubation piece 40 includes an abutment surface 41 correspondingly engaged on both sides of the cut-open part. The hollow intubation body 10 is in the retracted state when two abutment surfaces 41 are not engaged together (as shown in FIG. 2), and the hollow intubation body 10 is in the expanded state when the two abutment surfaces 41 are engaged together (as shown in FIG. 3). Furthermore, the two abutment surfaces 41 are respectively provided with a snap-fit structure 411, and one snap-fit structure 411 is a convex part, and the other one snap-fit structure 411 is a concave part.

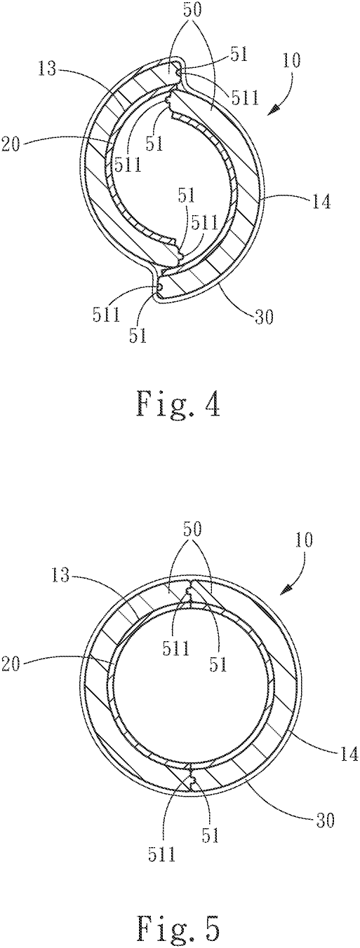

With reference to FIGS. 4 and 5, in another embodiment, the hollow intubation body 10 is composed of two half-tube bodies 50, and each end of the two half-tube bodies 50 includes an abutment surface 51 thereon, and the two half-tube bodies 50 are arranged crosswise so that the hollow intubation body 10 is in the retracted state (as shown in FIG. 4). The two half-tube bodies 50 can be engaged with each other with the abutment surface 51 on each end thereof so that the hollow intubation body 10 is in the expanded state (as shown in FIG. 5). In addition, a snap-fit structure 511 is provided at each end of two half-tube bodies, wherein the snap-fit structure 511 provided at one end is a convex part, and the snap-fit structure 511 provided at the other end is a concave part so that the convex part is engaged with the concave part.

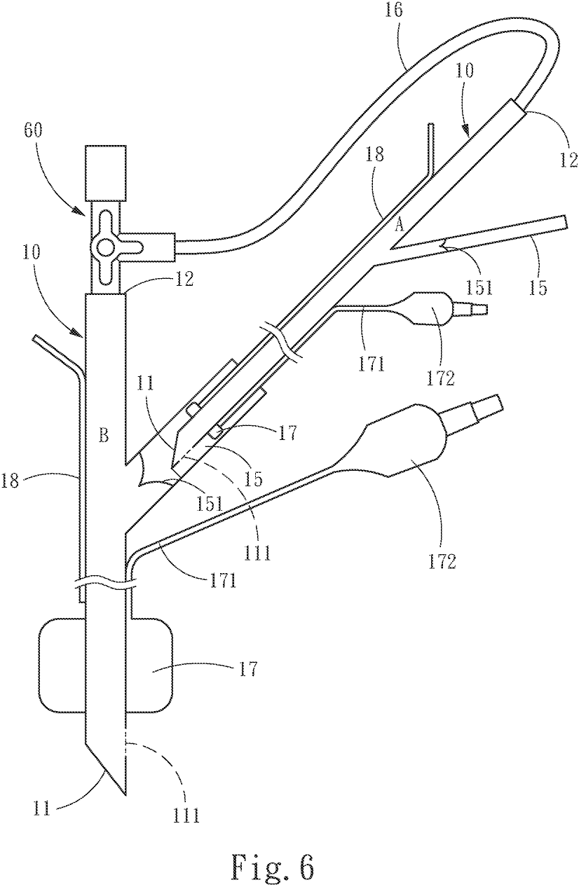

With reference to FIG. 6, a schematic diagram of the use and replacement of an endotracheal intubation according to another embodiment of the present invention. In the present embodiment, the hollow intubation body 10 is provided with a Murphy eye 111 adjacent to the front end 11, and the Murphy eye 111 serves as an alternative vent when the front end 11 is blocked; the hollow intubation body 10 further includes a bifurcated tube 15 located between the front end 11 and the tail end 12 and near the tail end 12, and the bifurcated tube 15 is provided with a one-way valve 151 allowing fluid flows toward the hollow intubation body 10. The use and replacement of the embodiment of the present invention are described below. For the convenience of identification, new and old hollow intubation bodies 10 are identified by reference numerals A and B on the drawings, respectively. When the old hollow intubation body 10 is used, the tail end 12 of the old hollow intubation body 10 is connected to an oxygen supply device (not shown) for supplying oxygen or air through a three-way outlet valve 60. When the old hollow intubation body 10 is to be replaced, the front end 11 of the new hollow intubation body 10 is inserted through the bifurcated tube 15 of the old hollow intubation body 10, in order to make the tail end 12 of the new hollow intubation body 10 being connected to the three-way outlet valve 60 through a breathing tube 16. In this way, the new hollow intubation body 10 in the inner retracted state is penetrated into the old hollow intubation body 10 in the expanded state through the bifurcated tube 15, and the new hollow intubation body 10 in the inner retracted state serves as a continuous oxygen supply channel during the process of changing intubations, so as to supply oxygen without interruption when changing intubations. As long as oxygen or air from the oxygen supply device is delivered from the new hollow intubation body 10 through switching of the three-way outlet valve 60, before the old hollow intubation body 10 is withdrawn, uninterrupted use of oxygen can be achieved.

In addition, a balloon 17 is provided outside the hollow intubation body 10, a blowing/exhausting port 171 is provided for blowing or exhausting air to control whether the balloon 17 is inflated, and a degree of inflation of the balloon 17 can be learned as a degree of inflation of the pilot balloon 172. When the hollow intubation body 10 is positioned, the balloon 17 is inflated and secured to the trachea (not shown). A sputum aspirating intubation 18 may also be provided outside the hollow intubation body 10 for aspirating secretions such as sputum to reduce the chance of infection.

Further, in the foregoing embodiments, the present invention may further comprise an inner membrane 20 disposed on the inner wall 13 and extending to the front end 11 and the tail end 12. In some embodiments, the inner membrane 20 is removed before insertion of the new hollow intubation body 10 into the old hollow intubation body 10 to remove sources of infection such as blood, mucous membranes, sputum, etc. which may reduce the risk of infection.

Another aspect of the present invention may include an elastomeric sleeve 30 surrounding the hollow intubation body 10 and extending to the front end 11 and the tail end 12. In some embodiments, the elastomeric sleeve 30 is used as a protective film. In the process that the two abutment surfaces 41 of the longitudinally sectioned intubation piece 40 are engaged, or in the process that the two half-tube bodies 50 are engaged with each other, the throat, vocal cords and trachea can be prevented from being pinched, resulting in additional injury.

In view of the foregoing, the endotracheal intubation according to the present invention can be mounted in a trachea of a patient, and when the endotracheal intubation needs to be replaced, the hollow intubation body of the new endotracheal intubation is directly put into the old hollow intubation body while being in the retracted state, then the old hollow intubation body is removed, and finally the new hollow intubation body is changed into the expanded state, that is, the replacement operation of the endotracheal intubation is completed.

The advantages of the present invention over the prior art include at least the following. (1). In order to use the old hollow intubation body as a protective layer, which does not contact the throat, vocal cords and trachea when inserting the new endotracheal intubation, the injury of the throat, vocal cords and trachea can be avoided and the replacement speed can be accelerated to meet the needs in use. (2). The risk of infection may be reduced by providing the inner membrane to remove sources of infection such as blood, mucous membranes, sputum, etc. (3). The provision of the flexible sleeve avoids pinching the throat, vocal cords and trachea. (4). Through the arrangement of the bifurcated tube, the hollow intubation body in the retracted state can be penetrated into the hollow intubation body in the expanded state through the bifurcated tube, and the hollow intubation body in the inner retracted state can serve as a continuous oxygen supply channel so as to achieve uninterrupted oxygen supply when changing intubations. (5). When the old hollow intubation is removed, sputum in trachea, throat and oral cavity can be sucked away through the sputum aspirating intubation, so as to reduce the probability of bacterial growth and pneumonia.

What is claimed is:

1. An endotracheal intubation for preventing injury to throat, vocal cords, and trachea, comprising:
a hollow intubation body with a variable outer diameter dimension, comprising a front end, a tail end, an inner wall and an outer wall, the front end and the tail end located at two opposite ends of the hollow intubation body, and the inner wall and the outer wall located at two opposite inner and outer sides of the hollow intubation body, and the hollow intubation body having a retracted state with a smaller outer diameter and an expanded state with a larger outer diameter, and the hollow intubation body in the retracted state being able to be changed to the expanded state, and the hollow intubation body in the retracted state being able to penetrate into another hollow intubation body in the expanded state.

2. The endotracheal intubation as claimed in claim 1, wherein the hollow intubation body further comprises a bifurcated tube between the front end and the tail end and near the tail end, and the bifurcated tube is provided with a one-way valve providing fluid to flow toward the hollow intubation body.

3. The endotracheal intubation as claimed in claim 1, wherein the endotracheal intubation comprises a flexible intubation sleeve covering the hollow intubation body and extending to the front end and the tail end.

4. The endotracheal intubation as claimed in claim 1, wherein the endotracheal intubation comprises an inner membrane disposed on the inner wall and extending to the front end and the tail end.

5. The endotracheal intubation as claimed in claim 1, wherein the hollow intubation body is a longitudinal sectioned intubation piece which is curved, the longitudinal sectioned intubation piece comprises two abutment surfaces respectively on both sides of the longitudinal sectioned intubation piece and are able to be correspondingly engaged, and wherein the hollow intubation body is in the retracted state when the two abutment surfaces are not correspondingly engaged, and the hollow intubation body is in the expanded state when the abutment surfaces are correspondingly engaged.

6. Then endotracheal intubation as claimed in claim 5, wherein the two abutment surfaces are each provided with a snap-fit structure.

7. The endotracheal intubation as claimed in claim 1, wherein the hollow intubation body is composed of two half-tube bodies, each end of the two half-tube bodies includes an abutment surface thereon, and the two half-tube bodies are arranged crosswise so that the hollow intubation body is in the retracted state, and two ends of the two half-tube bodies are engaged with each other with the abutment surface on each end so that the hollow intubation body is in the expanded state.

8. The endotracheal intubation according to claim 7, wherein a snap-fit structure is provided at each end of two half-tube bodies.
